(19) United States
(12) Patent Application Publication
(10) Pub. No.: US 2014/0135521 A1
(43) Pub. Date: May 15, 2014

(54) METHOD FOR PRODUCING ALPHA-HYDROXYCARBOXYLIC ACID ESTERS

(71) Applicants: Martin Koestner, Darmstadt (DE); Willi Ploesser, Seeheim-Jugenheim (DE); Alexander May, Seeheim-Jugenheim (DE)

(72) Inventors: Martin Koestner, Darmstadt (DE); Willi Ploesser, Seeheim-Jugenheim (DE); Alexander May, Seeheim-Jugenheim (DE)

(73) Assignee: Evonik Roehm GmbH, Darmstadt (DE)

(21) Appl. No.: 14/129,811

(22) PCT Filed: Jul. 3, 2012
(86) PCT No.: PCT/EP2012/062870

§ 371 Date: Dec. 27, 2013

(30) Foreign Application Priority Data
Aug. 19, 2011 (DE) 10 2011 081 256.3

(51) Int. Cl. C07C 51/06

(52) U.S. Cl. CPC C07C 51/06
USPC 560/179

(57) ABSTRACT

The present invention relates to a continuous process for preparing alpha-hydroxycarboxylic esters by reacting at least one alpha-hydroxycarboxamide present in the liquid phase with an alcohol in the presence of a catalyst, which is characterized in that the resulting alpha-hydroxycarboxylic ester is at least partly separated from the reaction mixture via the gas phase.

The present invention relates to processes for preparing alpha-hydroxycarboxylic esters.

Alpha-hydroxycarboxylic esters are valuable intermediates in the industrial-scale synthesis of acrylic esters and methacrylic esters, referred to hereinafter as alkyl (meth)acrylates. Alkyl (meth)acrylates are used in large amounts for preparation of polymers, for example polymethyl methacrylate.

An overview of the standard processes for preparing (meth)acrylic esters can be found in the literature, such as Weissermel, Arpe “Industrielle organische Chemie” [Industrial Organic Chemistry], VCH, Weinheim 1994, 4th edition, p. 305 ff. or Kirk Othmer “Encyclopedia of Chemical Technology”, 3rd edition, Vol. 15, page 357.

When the aim is the synthesis of methacrylic esters, for example methyl methacrylate, methyl 2-hydroxyisobutyrate (=MHIB), as the alpha-hydroxycarboxylic ester, is a central intermediate for preparation thereof.

The preparation of alpha-hydroxycarboxylic esters via the reaction of an alcohol with an alpha-hydroxycarboxamide is detailed by way of example in the publication DE-A-24 54 497. This publication describes the use of lead compounds in order to catalyse the reaction. In this context, mention is also made of continuous processes, but without providing a technical solution in which the products are obtained with high efficiency.

Furthermore, the document DE-A-25 28 524 describes processes for preparing alpha-hydroxycarboxylic esters. In this context, various catalysts are used, which include lanthanum compounds among others. Although DE-A-25 28 524 also mentions that the processes described can be performed continuously, this publication also does not provide a satisfactory solution to the problems which occur here.

A process of this type is known from EP 0 945 423. Here, a process for preparing alpha-hydroxycarboxylic esters is disclosed, which comprises the steps of reacting an alpha-hydroxycarboxamide and an alcohol in the presence of a catalyst in a liquid phase, while the ammonia concentration in the reaction solution is kept at 0.1% by weight.

For this reason, ammonia which forms is removed very substantially from the reaction solution. To this end, the reaction solution is heated to boiling, and/or a stripping gas, i.e. an inert gas, is bubbled through the reaction solution.

The disadvantages of the process disclosed in EP 0 945 423 for the preparation of alpha-hydroxycarboxylic esters by alcoholysis of corresponding alpha-hydroxy-carboxamides can be summarized as follows:
i. Simply distilling off the ammonia under conditions according to a process variant disclosed in EP 0 945 423 is not very effective. The implementation of this proposal requires an extremely effective separating column and hence an exceptional level of technical complexity.
ii. When an inert stripping gas is used additionally or exclusively, the effectiveness of the ammonia removal is improved, but at the expense of a further process component, the handling of which means additional complexity.
iii. When alpha-hydroxyisobutyramide and methanol are used as reactants, ammonia and residual methanol formed under the conditions disclosed in EP 0 945 423 can be separated from one another only with very great difficulty.

The fact that it is almost always necessary to use an inert gas for ammonia removal and the associated additional handling of a further stream (stripping gas/ammonia separation) make the procedure proposed economically relatively uninteresting, which is also reflected by the lack of an industrial implementation of the process disclosed to date.

A process improved over the methods detailed above is described in publication DE-A-10 2007 011706. In this process, the reaction of alpha-hydroxyisobutyramide with methanol is performed at a relatively high pressure, and the resulting methyl 2-hydroxyisobutyrate is passed out of the reactor, optionally together with residues of the alpha-hydroxyisobutyramide used. Even though this process can be performed much less expensively compared to the previously known methods and the products are obtained with very high selectivities, there is a continuing need for an improved process for preparing alpha-hydroxycarboxylic esters.

In view of the prior art, it was thus an object of the present invention to provide processes for preparing alpha-hydroxycarboxylic esters, which conserve energy and resources and can thus be performed in a simple and inexpensive manner.

It was a further object of the invention to provide a process in which the alpha-hydroxycarboxylic esters can be obtained very selectively.

It was a further object of the present invention to provide a process for preparing alpha-hydroxycarboxylic esters, in which only small amounts of by-products, if any, are obtained. At the same time, the product was to be obtained in maximum yields and, viewed overall, with minimum energy consumption.

It was another object of the present invention to provide processes which can be performed with plants which require a lower level of capital costs and maintenance expenditure than the plants needed for performance of the processes described in DE-A-10 2007 011706.

These objects, and further objects which are not stated explicitly but are immediately derivable or discernible from the connections discussed herein by way of introduction, are achieved by processes having all the features of claim 1. Appropriate modifications to the processes according to the invention are protected in the dependent claims which refer back to claim 1.

The present invention accordingly provides a continuous process for preparing alpha-hydroxycarboxylic esters by reacting at least one alpha-hydroxycarboxamide present in the liquid phase with an alcohol in the presence of a catalyst, which is characterized in that the resulting alpha-hydroxycarboxylic ester is at least partly separated from the reaction mixture via the gas phase.

The process according to the invention can be performed inexpensively, especially with a low energy requirement. At the same time, the catalysts used for alcoholysis of the alpha-hydroxycarboxamide can be used over a long period without any decrease in selectivity or activity. In this respect, the catalysts have a long service life.

At the same time, the formation of by-products is unusually low. In addition, especially taking account of the high selectivity, high conversions are achieved.

The process of the present invention also has an extremely low tendency to formation of by-products.

Furthermore, performance of the present process does not require costly plants associated with very high capital and maintenance costs.

The process according to the invention affords the alpha-hydroxycarboxylic esters in high yields and purities.

Finally, the process of the present invention can particularly advantageously be performed on the industrial scale.

In the process of the invention, alpha-hydroxycarboxylic esters are prepared by the reaction between the alpha-hydroxycarboxamide and alcohol reactants in the presence of a catalyst.

The alpha-hydroxycarboxamides usable in the reaction of the invention include typically all of those carboxamides which have at least one hydroxyl group in the alpha position to the carboxamide group.

Carboxamides in turn are common knowledge in the technical field. Typically, these are understood to mean compounds having groups of the formula —CONR′R″ in which R′ and R″ are each independently hydrogen or a group having 1-30 carbon atoms, which in particular comprises 1-20, preferably 1-10 and especially 1-5 carbon atoms, particular preference being given to amides where R′ and R″ are hydrogen. The carboxamide may comprise 1, 2, 3, 4 or more groups of the formula —CONR′R″. These include in particular compounds of the formula R(—CONR′R″)ₙ in which the R radical is a group having 1-30 carbon atoms, which in particular has 1-20, preferably 1-10, especially 1-5 and more preferably 2-3 carbon atoms, R′ and R″ are each as defined above and n is an integer in the range of 1-10, preferably 1-4 and more preferably 1 or 2.

The expression “group having 1 to 30 carbon atoms” denotes radicals of organic compounds having 1 to 30 carbon atoms. In addition to aromatic and heteroaromatic groups, it also includes aliphatic and heteroaliphatic groups, for example alkyl, cycloalkyl, alkoxy, cycloalkoxy, cycloalkylthio and alkenyl groups. The groups mentioned may be branched or unbranched.

According to the invention, aromatic groups denote radicals of mono- or polycyclic aromatic compounds having preferably 6 to 20, especially 6 to 12, carbon atoms.

Heteroaromatic groups denote aryl radicals in which at least one CH group has been replaced by N and/or at least two adjacent CH groups have been replaced by S, NH or O.

Aromatic or heteroaromatic groups preferred in accordance with the invention derive from benzene, naphthalene, biphenyl, diphenyl ether, diphenylmethane, diphenyl-dimethylmethane, bisphenone, diphenyl sulphone, thiophene, furan, pyrrole, thiazole, oxazole, imidazole, isothiazole, isoxazole, pyrazole, 1,3,4-oxadiazole, 2,5-diphenyl-1,3,4-oxadiazole, 1,3,4-thiadiazole, 1,3,4-triazole, 2,5-diphenyl-1,3,4-triazole, 1,2,5-triphenyl-1,3,4-triazole, 1,2,4-oxadiazole, 1,2,4-thiadiazole, 1,2,4-triazole, 1,2,3-triazole, 1,2,3,4-tetrazole, benzo[b]thiophene, benzo[b]furan, indole, benzo[c]thiophene, benzo[c]furan, isoindole, benzoxazole, benzothiazole, benzimidazole, benzisoxazole, benzisothiazle, benzopyrazole, benzothiadiazole, benzotriazole, dibenzofuran, dibenzothiophene, carbazole, pyridine, bipyridine, pyrazine, pyrazole, pyrimidine, pyridazine, 1,3,5-triazine, 1,2,4-triazine, 1,2,4,5-triazine, tetrazine, quinoline, isoquinoline, quinoxaline, quinazoline, cinnoline, 1,8-naphthyridine, 1,5-naphthyridine, 1,6-naphthyridine, 1,7-naphthyridine, phthalazine, pyridopyrimidine, purine, pteridine or quinolizine, 4H-quinolizine, diphenyl ether, anthracene, benzopyrrole, benzooxathiadiazole, benzooxadiazole, benzopyridine, benzopyrazine, benzopyrazidine, benzopyrimidine, benzotriazine, indolizine, pyridopyridine, imidazopyrimidine, pyrazinopyrimidine, carbazole, aciridine, phenazine, benzoquinoline, phenoxazine, phenothiazine, acridizine, benzopteridine, phenanthroline and phenanthrene, each of which may also optionally be substituted.

The preferred alkyl groups include the methyl, ethyl, propyl, isopropyl, 1-butyl, 2-butyl, 2-methylpropyl, tert-butyl, pentyl, 2-methylbutyl, 1,1-dimethylpropyl, hexyl, heptyl, octyl, 1,1,3,3-tetramethylbutyl, nonyl, 1-decyl, 2-decyl, undecyl, dodecyl, pentadecyl and the eicosyl group.

The preferred cycloalkyl groups include the cyclopropyl, cyclobutyl, cyclopentyl, cyclohexyl, cycloheptyl and the cyclooctyl group, each of which is optionally substituted by branched or unbranched alkyl groups.

The preferred alkenyl groups include the vinyl, allyl, 2-methyl-2-propenyl, 2-butenyl, 2-pentenyl, 2-decenyl and the 2-eicosenyl group.

The preferred heteroaliphatic groups include the aforementioned preferred alkyl and cycloalkyl radicals in which at least one carbon unit has been replaced by O, S or an NR⁸ or NR⁸R⁹ group, and R⁸ and R⁹ are each independently an alkyl group having 1 to 6 carbon atoms, an alkoxy group having 1 to 6 carbon atoms or an aryl group.

Most preferably in accordance with the invention, the carboxamides have branched or unbranched alkyl or alkoxy groups having 1 to 20 carbon atoms, preferably 1 to 12, appropriately 1 to 6, in particular 1 to 4 carbon atoms, and cycloalkyl or cyclo-alkyloxy groups having 3 to 20 carbon atoms, preferably 5 to 6 carbon atoms.

The R radical may have substituents. The preferred substituents include halogens, especially fluorine, chlorine, bromine, and alkoxy or hydroxyl radicals.

The alpha-hydroxycarboxamides may be used in the process of the invention individually or as a mixture of two or three or more different alpha-hydroxy-carboxamides. Particularly preferred alpha-hydroxycarboxamides include alpha-hydroxyisobutyramide and/or alpha-hydroxyisopropionamide.

It is also of particular interest, in a modification of the process according to the invention, to use those alpha-hydroxycarboxamides which are obtainable by cyanohydrin synthesis from ketones or aldehydes and hydrogen cyanide. In a first step, the carbonyl compound, for example a ketone, especially acetone, or an aldehyde, for example acetaldehyde, propanal, butanal, is reacted with hydrogen cyanide to give the particular cyanohydrin. Particular preference is given to reacting acetone and/or acetaldehyde in a typical manner using a small amount of alkali or of an amine as a catalyst. In a further step, the cyanohydrin thus obtained is reacted with water to give the alpha-hydroxycarboxamide.

This reaction is typically performed in the presence of a catalyst. Suitable catalysts for this purpose are in particular manganese oxide catalysts, as described, for example, in EP-A-0945429, EP-A-0561614 and EP-A-0545697. The manganese oxide may be used in the form of manganese dioxide, which is obtained by treating manganese sulphate with potassium permanganate under acidic conditions (cf. Biochem. J., 50, p. 43 (1951) and J. Chem. Soc., 1953, p. 2189, 1953) or by electrolytic oxidation of manganese sulphate in aqueous solution. In general, the catalyst is used in many cases in the form of powder or granule with a suitable particle size. In addition, the catalyst may be applied to a support. In particular, it is also possible to use so-called slurry reactors or fixed bed reactors, which may also be operated as a trickle bed and are described, inter alia, in EP-A-956 898. In addition, the hydrolysis reaction may be catalysed by enzymes. The suitable enzymes include nitrile hydratases. This reaction is described by way of example in “Screening, Characterization and Application of Cyanide-resistant Nitrile Hydratases” Eng. Life. Sci. 2004, 4, No. 6. In addition, the hydrolysis reaction can be catalysed by acids, especially sulphuric acid. This is detailed, inter alia, in JP Hei 4-193845.

In addition, the processes detailed above for preparing alpha-hydroxycarboxamides are detailed, inter alia, in WO 2009/130075 A2, and the processes detailed in this publication are inserted into the present application by reference for disclosure purposes.

The alcohols usable successfully in processes of the invention include all alcohols which are familiar to those skilled in the art and precursor compounds of alcohols which, under the given conditions of pressure and temperature, are capable of reacting with the alpha-hydroxycarboxamides in the manner of an alcoholysis. Preference is given to converting the α-hydroxycarboxamide by alcoholysis with an alcohol, which comprises preferably 1-10 carbon atoms, more preferably 1 to 5 carbon atoms. Preferred alcohols include methanol, ethanol, propanol, butanol, especially n-butanol and 2-methyl-1-propanol, pentanol, hexanol, heptanol, 2-ethyl-hexanol, octanol, nonanol and decanol. The alcohol used is more preferably methanol and/or ethanol, methanol being very particularly appropriate. It is also possible in principle to use precursors of an alcohol. For example, alkyl formates may be used. Methyl formate or a mixture of methanol and carbon monoxide are especially suitable.

Preference is further given to processes which are characterized in that the alpha-hydroxycarboxamide used is hydroxyisobutyramide and the alcohol used is methanol.

The reaction according to the invention takes place in the presence of a catalyst. The reaction can be accelerated, for example, by basic catalysts. These include homogeneous catalysts and heterogeneous catalysts.

Catalysts of very particular interest for the performance of the process according to the invention are lanthanoid compounds.

Lanthanoid compounds refer to compounds of La, Ce, Pr, Nd, Pm, Sm, Eu, Gd, Td, Dy, Ho, Er, Tm, Yb and/or Lu. Preference is given to using a lanthanoid compound which comprises lanthanum.

Preferred lanthanoid compounds are salts which are preferably present in the oxidation state of 3.

Particularly preferred water-stable lanthanoid compounds are La(NO₃)₃ and/or LaCl₃. These compounds may be added to the reaction mixture as salts or be formed in situ.

Further homogeneous catalysts usable successfully in the present invention include alkali metal alkoxides and organometallic compounds of titanium, tin and aluminium. Preference is given to using a titanium alkoxide or tin alkoxide, for example titanium tetraisopropyloxide or tin tetrabutyloxide.

A particular process variant includes the use, as the catalyst, of a soluble metal complex which comprises titanium and/or tin and the alpha-hydroxycarboxamide.

Another specific modification of the process of the invention envisages that the catalyst used is a metal trifluoromethanesulphonate. Preference is given to using a metal trifluoromethanesulphonate in which the metal is selected from the group consisting of the elements in groups 1, 2, 3, 4, 11, 12, 13 and 14 of the periodic table.

Among these, preference is given to using those metal trifluoromethanesulphonates in which the metal corresponds to one or more lanthanoids.

In addition to the preferred variants of homogeneous catalysis, processes using heterogeneous catalysts are also appropriate. The heterogeneous catalysts usable successfully include magnesium oxide, calcium oxide and basic ion exchangers and the like.

For example, preference may be given to processes in which the catalyst is an insoluble metal oxide which contains at least one element selected from the group consisting of Sb, Sc, V, La, Ce, Ti, Zr, Hf, V, Nb, Ta, Cr, Mo, W, Tc, Re, Fe, Co, Ni, Cu, Al, Si, Sn, Pb and Bi.

Alternatively, preference may be given to processes in which the catalyst used is an insoluble metal selected from the group consisting of Ti, Zr, Hf, V, Nb, Ta, Cr, Mo, W, Fe, Co, Ni, Cu, Ga, In, Bi and Te.

The preferred heterogeneous catalysts include especially catalysts based on ZrO₂ and/or Al₂O₃. Particularly preferred catalysts of this type are described in detail more particularly in JP 6-345692, the catalysts detailed in JP 6-345692 being incorporated into the present application by reference for disclosure purposes.

The ammonia released in preferred variants of the process of the present invention can, for example, be recycled in a simple manner to an overall process for preparing alkyl (meth)acrylates. For example, ammonia can be reacted with methanol to give hydrocyanic acid. This is detailed, for example, in EP-A-0941984. In addition, the hydrocyanic acid can be obtained from ammonia and methane by the BMA or Andrussow process, these processes being described in Ullmann's Encyclopaedia of Industrial Chemistry 5th edition on CD-ROM, under “Inorganic Cyano Compounds”. The ammonia can likewise be recycled into an ammoxidation process, for example the industrial scale synthesis of acrylonitrile from ammonia, oxygen and propene. The acrylonitrile synthesis is described under “Sohio Process” in Industrial Organic Chemistry by K. Weisermehl and H.-J. Arpe on page 307 ff.

According to the invention, the resulting alpha-hydroxycarboxylic ester is at least partly removed from the reaction mixture via the gas phase. In a particular configuration of the process, preferably at least 60% by weight, especially at least 80% by weight, more preferably at least 90% by weight and most preferably at least 95% by weight of the resulting alpha-hydroxycarboxylic ester can be removed from the reaction mixture via the gas phase. Accordingly, the process is preferably executed in such a way that a maximum proportion of the product is converted to the gas phase. This aim can be achieved especially through the selection of the reactor, through the choice of pressure and temperature, and the gas volume in the course of operation of the reactor, especially in relation to the overall volume or the liquid volume thereof.

The process according to the invention is executed continuously. Continuous processes are notable in that all reactants are constantly introduced into the reactor and all products removed from the reactor, such that the reaction can be performed over an indeterminate period. This is not affected by interruptions which are necessary due to maintenance or cleaning measures.

In this context, the reaction can be executed in such a way that the alpha-hydroxycarboxylic ester is separated in a separate step from the nitrogen compound released from the reaction mixture. Surprising advantages arise, however, in embodiments which are characterized in that the alpha-hydroxycarboxylic ester is separated from the reaction mixture together with the nitrogen compound released, preferably ammonia released. Advantages arise especially through processes in which the molar ratio of alpha-hydroxycarboxylic ester to ammonia during the separation of these components from the reaction mixture is in the range from 2:1 to 1:2, more preferably 1.2:1 to 1:1.2.

Of particular interest are processes in which the concentration of alpha-hydroxycarboxylic ester in the liquid phase of the reaction mixture is preferably kept less than 30% by weight, especially less than 20% by weight, preferably less than 10% by weight and more preferably less than 5% by weight.

The molar ratio of alpha-hydroxycarboxylic ester to alpha-hydroxycarboxamide in the liquid phase of the reaction mixture is preferably less than 1, more preferably less than 0.8 and more preferably less than 0.1.

Surprising advantages with regard to the productivity of the process, especially with regard to the costs for performance thereof, can be achieved by introducing the alcohol into the reaction mixture as a gas.

The type of reactor for performance of the present process is not restricted. Preference is given, however, to using those reactors into which relatively large amounts of gas can be introduced or removed. Preference is accordingly given to using multiphase reactors for performance of the present process.

It is possible here to use multiphase reactors in which a gas is introduced in countercurrent relative to the liquid phase. These reactors include reactors based on sparged stirred tanks or cascades. In addition, a gas can be passed in countercurrent to the liquid through a tray column or column containing random packings, and this arrangement is suitable for performance of the present process.

In a preferred embodiment, the alcohol can be introduced into the reaction mixture in cocurrent. This can preferably be done in a reactor in which the alcohol is supplied as a gas in cocurrent. Particularly suitable reactors include trickle bed reactors, bubble column reactors, jet scrubbers and falling-film reactors, particular preference being given to trickle bed reactors and falling-film reactors, or the combination of trickle bed reactors and falling-film reactors.

Trickle bed reactors are generally understood to mean reactors which are typically, but not necessarily, operated in cocurrent of gas and liquid by means of interface-generating internals or beds. Trickle bed reactors are notable for their narrow residence time distribution for gas and liquid phase. Trickle bed reactors can be designed as fixed bed columns or columns with random packing.

Falling-film reactors enable simple and effective supply and removal of heat, which is found to be advantageous especially in reactions with high exothermicity or in the case of phase transition of a reactant or product.

More detailed descriptions can be found in the specialist literature (e.g. Ullmanns Encyklopadie der technischen Chemie, Volume 3, 4^{th} edition p. 357ff and p. 500ff).

For execution of the present process, preference is given especially to multiphase reactors which are notable for a high gas content in the reactor volume. Particular reactors accordingly feature a gas content which is preferably at least 50% by volume, more preferably at least 60% by volume. The quotient of mass transfer area of the reactor which converts the alpha-hydroxycarboxylic ester to the gas phase to the reactor volume may preferably be at least 100 m⁻¹, more preferably at least 500 m⁻¹.

The generation of gas-liquid interfaces in multiphase reactors can be effected in a different manner according to the reactor type. As well as the introduction of energy in the form of kinetic energy or pressure energy, the use of structured internals is especially appropriate. The structured internals include random packings such as Raschig rings, lnterpak, or structured packings such as Mellapak, etc. to Katapak, or appropriately a heterogeneous catalyst in a corresponding appropriate shape.

The liquid which remains after the reaction with the alcohol and the removal of the alpha-hydroxycarboxylic ester may contain alpha-hydroxycarboxamide. This remaining reactant can be worked up by customary purification processes. Processes of particular interest, however, are those in which the alpha-hydroxycarboxamide is circulated in the reactor. It is possible here to remove by-products with a high boiling point from the circuit by means of an evaporator, for example by means of a thin-film evaporator.

The vapour phase removed from the reactor may, as well as the products, also comprise unconverted alcohol. As well as customary purification processes, especially distillation processes, the recycling of the unconverted alcohol, either in liquid or vaporous form, is of particular interest.

Processes of particular interest are therefore those in which the reaction is performed preferably at a temperature in the range of 50-300° C., more preferably in the range from 150 to 200° C.

The pressure at which the conversion takes place is not critical per se. Since the boiling temperature of the alpha-hydroxycarboxylic ester is, however, dependent thereon and the alpha-hydroxycarboxylic ester has to be converted to the gas phase, the pressure has to be selected as a function of temperature, and low temperatures result in relatively low pressures. The reaction can preferably be performed at a pressure in the range from 0.01 to 20 bar, more preferably in the range from 0.1 to 10 bar.

The above measures allow the reaction to be performed at relatively low temperatures and pressures, which achieves particularly high selectivities and very high yields of substance of value. This also makes the apparatus for performance of the reaction under these conditions particularly simple and hence inexpensive.

This way of conducting the reaction is found to be particularly advantageous with regard to the energy consumption per mole of alpha-hydroxycarboxylic ester and ammonia formed and purified as a pure substance. The energy consumption is essentially determined by the conversion of methanol per pass.

### EXAMPLE 1

In a continuous laboratory test plant consisting of a reactant metering system, a trickle bed reactor designed as a column with random packing (ID 100 mm, I 1000 mm, Interpak 10 mm random packings) with liquid circulation and vapour phase removal, and also a production condensation system, vaporous methanol and alpha-hydroxyisobutyramide supplied as a melt were converted with the aid of a catalyst soluble in the liquid phase over 48 h. The catalyst used was La(NO₃)×6H₂O with a concentration of 2% by weight in the liquid phase. The temperature of liquid circulation was 180° C.; the pressure in the reactor was set to 800 mbar. The vapour phase was condensed completely and continuously, and the composition was determined by gas chromatography and titration. The selectivity for methyl alpha-hydroxyisobutyrate based on methanol was 99.8%; the ammonia concentration in the condensate was 4.8% by weight. The conversion of methanol averaged over the experiment time was 12%.

### Comparative Example 1

In a laboratory test plant consisting of a reactant metering system and a continuous stirred tank reactor, 157 g/h of a methanol/catalyst mixture with a catalyst content of 0.8% by weight and 35 g/h of alpha-hydroxyisobutyramide were supplied over an experiment time of 48 h. The conversion was performed using La(NO₃)₃ as a catalyst in fully liquid phase at 60 bar at a temperature of 200° C. The product mixture formed was analysed by means of gas chromatography. The molar selectivity for methyl alpha-hydroxyisobutyrate based on alpha-hydroxyisobutyramide was 98.7%, while the selectivity for methyl hydroxyisobutyrate based on methanol was 99.2%. In the fully liquid product mixture, an ammonia concentration of 0.7% by weight was found. The average conversion of methanol was 1.8%.

### EXAMPLE 2

The trickle bed reactor used in Example 1 was modified in that a heterogeneous catalyst based on ZrO₂ (3 mm pellets) was used as the catalyst instead of random packings. Over a period of 48 h, vaporous methanol and alpha-hydroxyisobutyramide supplied as a melt were converted. The temperature of the liquid circulation was 170° C.; the pressure in the reactor was set to 800 mbar. The vapour phase was condensed fully and continuously and the composition was determined by gas chromatography and titration. The selectivity for methyl alpha-hydroxyisobutyrate based on methanol was 99.85%; the ammonia concentration in the condensate was 4.83% by weight. The mean conversion of methanol was 13%.

1. A continuous process for preparing an alpha-hydroxycarboxylic ester, comprising reacting, as a reaction mixture, an alpha-hydroxycarboxamide present in a liquid phase with an alcohol in the presence of a catalyst to obtain the alpha-hydroxycarboxylic ester,
wherein the alpha-hydroxycarboxylic ester is at least partly separated from the reaction mixture via a gas phase.

2. The process according to claim 1, wherein the alpha-hydroxycarboxylic ester is separated from the reaction mixture with release of ammonia.

3. The process according to claim 1, wherein at least 90% by weight of the alpha-hydroxycarboxylic ester is separated from the reaction mixture via the gas phase.

4. The process according to claim 1, wherein a concentration of alpha-hydroxycarboxylic ester in the liquid phase of the reaction mixture is less than 10% by weight.

5. The process according to claim 1, wherein a molar ratio of alpha-hydroxycarboxylic ester to alpha-hydroxycarboxamide in the liquid phase of the reaction mixture is less than 1.

6. The process according to claim 1, wherein the alcohol is introduced into the reaction mixture as a gas.

7. The process according to claim 1, wherein the reaction is performed in a multiphase reactor.

8. The process according to claim 7, wherein a gas content in the multiphase reactor is at least 50% by volume.

9. The process according to claim 7, wherein a quotient of mass transfer area of the reactor which converts the alpha-hydroxycarboxylic ester to the gas phase to a reactor volume is at least 100 m⁻¹.

10. The process according to claim 6, wherein alpha-hydroxycarboxamide is circulated in the reactor.

11. The process according to claim 10, wherein by-products having a high boiling point are removed from a circuit by means of a thin-film evaporator.

12. The process according to claim 1, wherein the catalyst is a heterogeneous catalyst.

13. The process according to claim 1, wherein the catalyst is a homogeneous catalyst.

14. The process according to claim 1, wherein the alcohol is at least one selected from the group consisting of α-hydroxyisobutyramide, α-hydroxyisopropionamide α-hydroxyisobutyramide, and methanol.

15. The process according to claim 1, wherein the reaction is performed at a temperature of from 50-300° C. and at a pressure of from 0.01 to 20 bar.

16. The process according to claim 1, wherein the catalyst is a heterogeneous catalyst based on ZrO₂, Al₂O₃, or both.

17. The process according to claim 1, wherein the catalyst is a homogeneous catalyst based on a lanthanoid compound.
